(19) United States
(12) Patent Application Publication
(10) Pub. No.: US 2017/0281387 A1
(43) Pub. Date: Oct. 5, 2017

(54) SLIP-RESISTANT STABILIZATION DEVICE AND METHODS OF USE

(71) Applicant: Blake A. MATHIE, Northville, MI (US)

(72) Inventor: Blake A. MATHIE, Northville, MI (US)

(21) Appl. No.: 15/477,566

(22) Filed: Apr. 3, 2017

Related U.S. Application Data
(60) Provisional application No. 62/317,041, filed on Apr. 1, 2016.

(51) Int. Cl. A61F 5/01

(52) U.S. Cl. CPC A61F 5/013; A61F 5/0102; A61H 1/0274

(57) ABSTRACT

An apparatus may include a wearable component having an interior surface and an exterior surface, with a slip-resistant material adhered to the interior surface, the wearable component separably attached to a connecting portion having a receiving element, and the receiving element separably attached to an attachment element removably affixed to an external support structure. A method of stabilization may include obtaining the apparatus, applying the wearable component to a portion of a body, attaching the attachment element to the external support structure, and separably attaching the connecting portion to the attachment element.

### CROSS-REFERENCE TO RELATED APPLICATIONS

This application claims priority to and benefit of U.S. Provisional Application Ser. No. 62/317,041, filed Apr. 1, 2016, entitled “Slip-Resistant Stabilization Device and Methods of Use,” which is hereby incorporated by reference in its entirety.

### BACKGROUND

Exoskeletal devices and other extracorporeal physical augmentations can be used to increase independence for subjects with muscle weakness, such as those with muscular dystrophy. Current exoskeletal devices are designed to assist those in the early stages of functional loss. Such devices may be passive or active, and serve to counteract gravity in order to increase subjects' active range of motion.

However, exoskeletal devices may be difficult for subjects to connect to portions of their body without assistance, particularly as their muscle weakness progresses. Further, traditional exoskeletal devices are bulky and cumbersome, with significant hardware visible to others. Therefore, there exists a need for a slim, easily concealed device that is easy for the user to attach to an external support with little to no assistance.

### SUMMARY

In some embodiments, an apparatus may include a wearable component having an interior surface and an exterior surface, with a slip-resistant material adhered to the interior surface, the wearable component separably attached to a connecting portion having a receiving element, and the receiving element separably attached to an attachment element removably affixed to an external support structure. In some embodiments, the wearable component may further comprise a strap. In certain embodiments, the strap may have a hook-and-loop surface. In some embodiments, the wearable component may be shaped to conform to a portion of a body. In certain embodiments, the connecting portion may be shaped to conform to the portion of the body.

In some embodiments, a method of stabilization may include obtaining an apparatus including a wearable component with an interior surface and an exterior surface, with a slip-resistant material adhered to the interior surface, the wearable component separably attached to a connecting portion having a receiving element, and the receiving element configured to receive an attachment element removably affixed to an external support structure, applying the wearable component to a portion of a body, attaching the attachment element to the external support structure, and separably attaching the connecting portion to the attachment element.

### BRIEF DESCRIPTION OF THE DRAWINGS

FIG. 1 depicts an illustrative embodiment of an apparatus on an arm in accordance with the present disclosure.

FIG. 2 depicts an illustrative embodiment of an apparatus on an arm in a flexed position in accordance with the present disclosure.

FIG. 3 depicts an alternative view of an apparatus on an arm in a flexed position in accordance with the present disclosure.

FIG. 4 depicts an illustrative embodiment of an apparatus on an arm in an extended position in accordance with the present disclosure.

FIG. 5 depicts an illustrative embodiment of an apparatus on an arm in an overhead extended position in accordance with the present disclosure.

FIG. 6 depicts an illustrative embodiment of a connecting portion, an attachment element, and a receiving element of an apparatus in accordance with the present disclosure.

FIG. 7 depicts an illustrative embodiment of a wearable component having a strap, in accordance with the present disclosure.

FIG. 8 depicts an illustrative embodiment of an interior surface of a wearable component having a strap, with a slip-resistant material adhered to the interior surface, in accordance with the present disclosure.

FIG. 9 depicts an illustrative embodiment of an exterior surface of a wearable component having a strap in accordance with the present disclosure.

FIG. 10 depicts an illustrative embodiment of a method of stabilization in accordance with the present disclosure.

### DETAILED DESCRIPTION

This disclosure is not limited to the particular systems, devices, and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein and in the appended claims and embodiments, the singular forms “a,” “an,” and “the” include plural references, unless the context clearly dictates otherwise. Thus, for example, reference to an “element” is a reference to one or more elements and equivalents thereof known to those skilled in the art, and so forth.

The term “body” as used herein includes, but is not limited to, the body of a human, a non-human vertebrate, and an animal such as a wild, domestic, or farm animal. As used herein, a portion of a body may include one or more portions of any body, including, but not limited to, a portion of an arm, a portion of a hand, a portion of a leg, a portion of a foot, a portion of a torso, a portion of a head, and a portion of a neck.

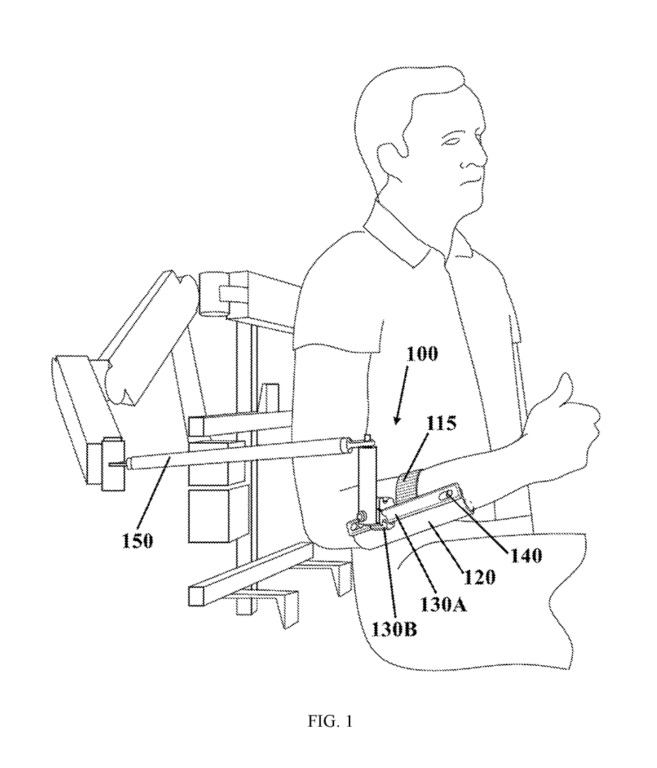

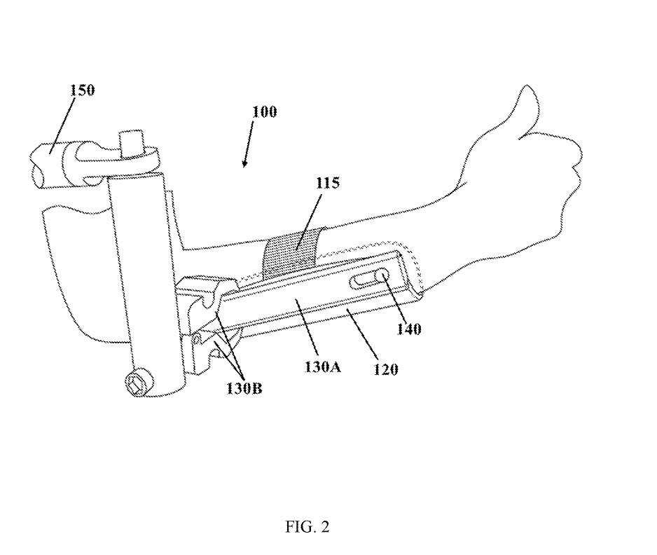

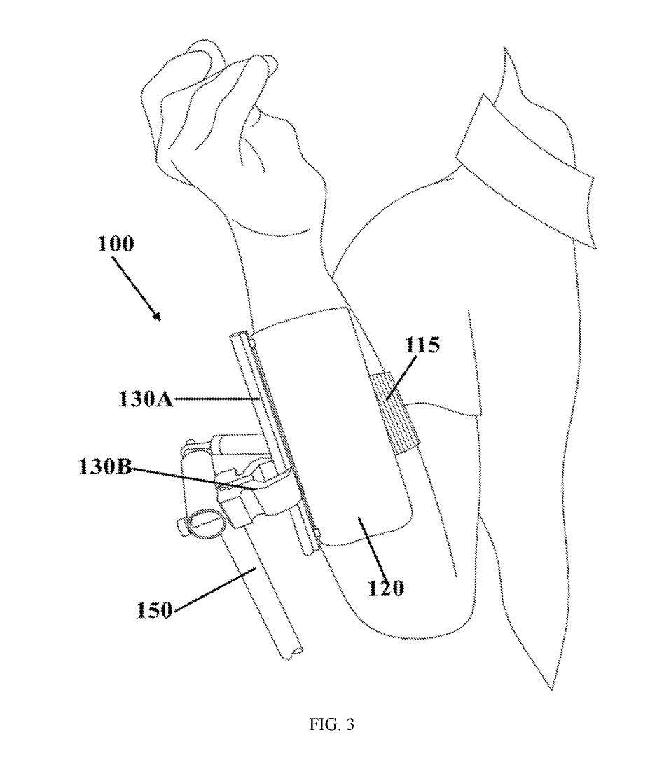

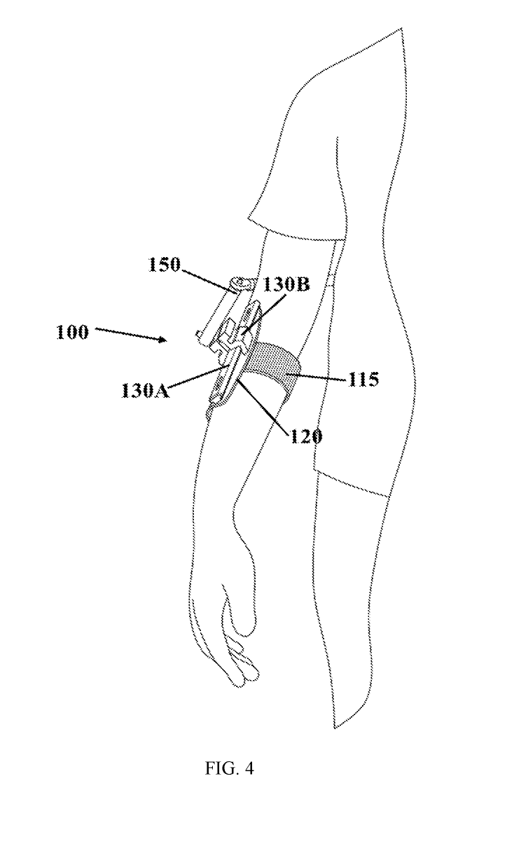

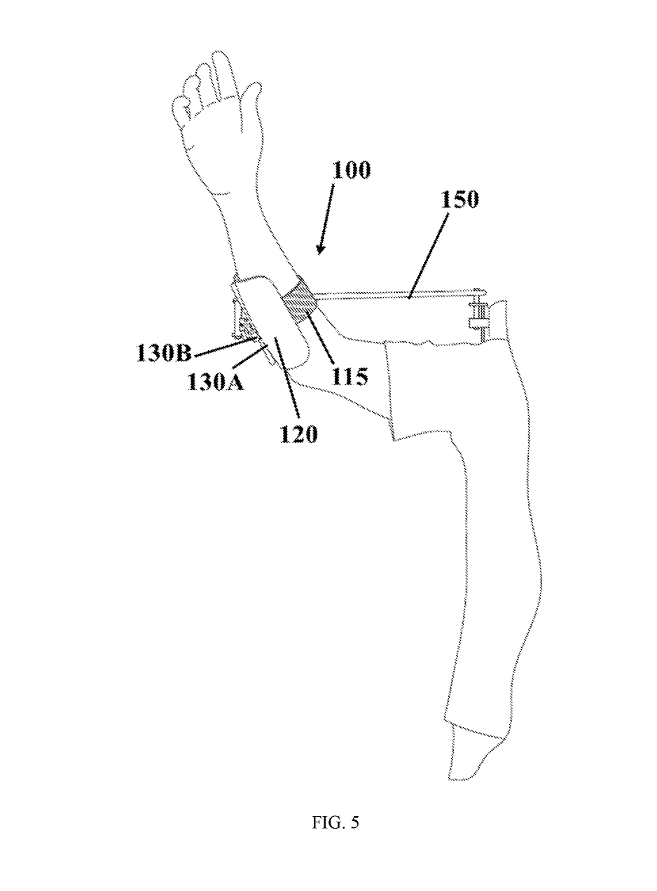

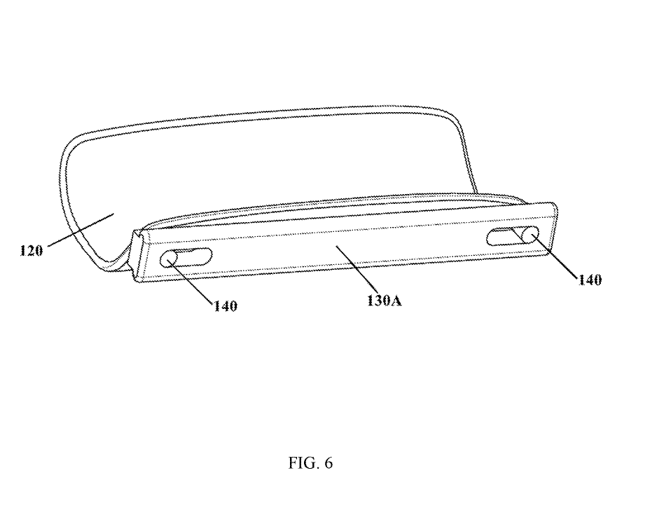

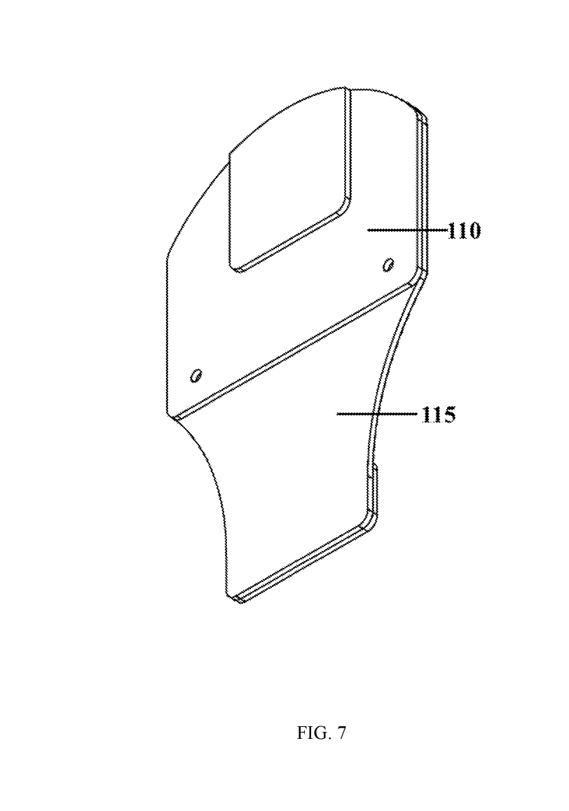

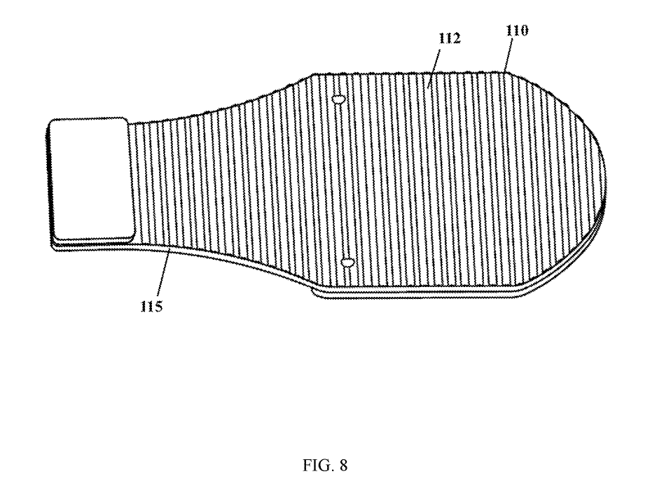

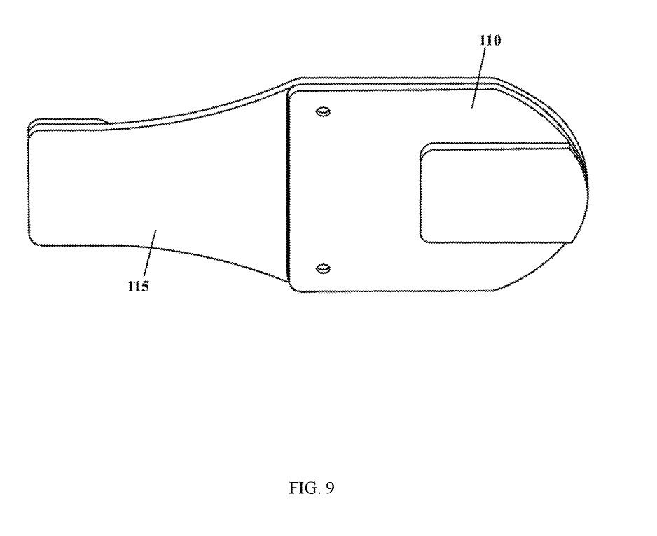

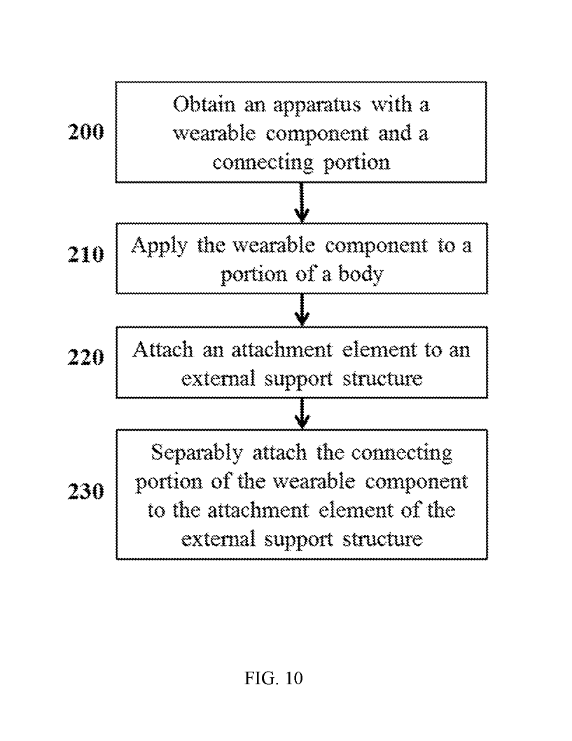

Embodiments disclosed herein relate to systems, devices, and methods for stabilizing a portion of a body. Various non-limiting embodiments of the apparatus are depicted in FIG. 1, FIG. 2, FIG. 3, FIG. 4, FIG. 5, FIG. 6, FIG. 7, FIG. 8, and FIG. 9. A non-limiting embodiment of the method described herein is depicted in FIG. 10.

### Apparatus

In some embodiments, an apparatus 100 may include a wearable component 110 and a connecting portion 120. The wearable component 110 may have an interior surface and an exterior surface, and the interior surface may include a slip-resistant material 112 adhered to it. In certain embodiments, the wearable component 110 may also include a strap 115, which may be used to secure the wearable component to a portion of a subject. The wearable component 110 may be separably attached to the connecting portion 120, and the connecting portion may further include one or more receiving elements 140. The receiving elements 140 may be configured to separably attach to an attachment element 130, and the attachment element 130 may be removably affixed to an external support structure 150. In some embodiments, one or more of the wearable component 110, the connecting portion 120, and the attachment element 130 may include one or more engagement elements (not shown). Exemplary embodiments of each element of such an apparatus are described in detail below.

### Wearable Component

In some embodiments, the wearable component 110 may comprise an elastomeric, form-fitting component. In certain embodiments, the wearable component 110 may comprise any material capable of being stretched. In some embodiments, the wearable component 110 may comprise any material capable of conforming to a portion of a body. In some embodiments, the wearable component 110 may comprise one or more elastomeric materials, such as polymers. In some embodiments, the polymers may be selected from the group consisting of neoprene, natural rubber, synthetic rubber, nylon, cotton, spandex, and any combinations thereof. In certain embodiments, the wearable component 110 may comprise a sleeve.

In other embodiments, the wearable component 110 may comprise a rigid polymer portion. In such embodiments, the polymer portion may comprise, for example, an air-cured polymer, a temperature-cured polymer, polyethylene terephthalate, polyethylene, polyvinyl chloride, polypropylene, polystyrene, fiberglass, carbon fiber, or combinations thereof. In certain embodiments, the rigid polymer portion may comprise a polymer that is capable of being custom-fitted, or molded, to a portion of a subject's body. In another embodiment, the wearable component 110 may further comprise a strap 115. In certain embodiments, the strap 115 may include a hook-and-loop surface. In some embodiments, the hook-and-loop surface of the strap 115 may be used to affix the strap to itself, after wrapping it around a portion of a subject's body.

In some embodiments, the wearable component 110 may have a slip-resistant material 112 adhered to its interior surface. In some embodiments, the slip-resistant material 112 may serve to prevent the wearable component 110 from moving excessively from its intended location on a portion of a body during the use of the apparatus 100. The slip-resistant material 112 may comprise any material that, when placed against the part of the body for which it is intended, has a coefficient of friction sufficient to resist slipping. In some embodiments, the slip-resistant material 112 may comprise silicone.

### Connecting Portion

In some embodiments, the connecting portion 120 of the apparatus 100 may comprise a substantially rigid material, such as a rigid polymer. In some embodiments, the connecting portion 120 may comprise a material selected from the group consisting of an air-cured polymer, a temperature-cured polymer, polyethylene terephthalate, polyethylene, polyvinyl chloride, polypropylene, polystyrene, fiberglass, carbon fiber, and combinations thereof. In certain embodiments, the connecting portion 120 may be form-fitted, or molded, to a particular portion of a subject's body. In some embodiments, the connecting portion 120 may be custom-molded to the portion of the subject's body.

The connecting portion 120 may serve to releasably connect the wearable component 110 to the attachment element 130 of the external support structure 150. In some embodiments, the connecting portion 120 may be removably engaged with the wearable component 110, using one or more engagement elements (not shown). In some embodiments, the connecting portion 120 may include one or more receiving elements 140. The receiving elements 140 may be configured to separably attach the connecting portion 120 to one or more attachment elements 130. In some embodiments, a receiving element 140 may include a clamp, a bracket, a bolt, a clip, a quick coupling construct, a protrusion, a magnet, or any combination thereof. FIG. 6 illustrates an embodiment of two receiving elements 140 of a connecting portion 120 separably attached to an attachment element 130A.

In some embodiments, the connecting portion 120 may be capable of separably attaching to the support member (not shown). In some embodiments, the shape of the connecting portion 120 may be designed to follow the shape of the support member (not shown), such that the connecting portion and the support member may be flush or nearly flush when attached.

In some embodiments, the receiving element 140 of the connecting portion 120 may be configured to attach to the engagement element (not shown) of the support member (not shown), such that the two elements may later be separated (i.e., separably attached). In some embodiments, the engagement element (not shown) and the support member (not shown) may independently comprise a metal or ferrous component that may engage the receiving element 140, which may comprise a magnet. In an alternative view of such an embodiment, the support member (not shown) may be made of a metal or ferrous material, which may act as the engagement element (not shown) for engaging the receiving element 140. In some embodiments, the receiving element 140 and the engagement element (not shown) may comprise, together or separately, a combination of one or more keyways (not shown) configured to engage one or more corresponding keys (not shown). Such keyways or keys may be present on either the support member or the connecting portion. The keyways and keys may act to resist rotation or translation of components of the apparatus 100 during use. In an illustrative embodiment, the keyway (not shown) is a recessed surface, and the key (not shown) is a protrusion on the support member (not shown). Although keyed surfaces, recessed areas, keys, and protrusions are described herein, other arrangements configured to resist rotation or translation would be apparent to this skilled in the art in view of this disclosure.

### Attachment Element

In some embodiments, the connecting portion 120 may have one or more receiving elements 140 separably attached to an attachment element 130. The attachment element 130 may itself be removably affixed to an external support structure 150. In some embodiments, the attachment element 130 may comprise a combination of components such as, for example, quick coupling constructs, screws, nails, bolts, clamps, bars, clips, protrusions, or combinations thereof. In certain embodiments, the attachment element 130 may comprise a rail 130A and a clamp 130B, as illustrated in FIG. 1, FIG. 2, FIG. 3, FIG. 4, and FIG. 5. In such embodiments, the rail 130A and the clamp 130B of the attachment element 130 may be in a fixed position with respect to one another. In other embodiments, the rail 130A and the clamp 130B of the attachment element 130 may be configured to slide, such that the clamp 130B slides along the rail 130A of the attachment element 130 across the subject's range of motion. In some embodiments, the attachment element 130 may be removably affixed to an external support structure 150. In some embodiments, the attachment element 130 may be configured to, for example, screw into or be bolted onto the external support structure 150.

### External Support Structure

In some embodiments, the external support structure 150 may comprise, for example, a portion of a chair or other device that may serve to stabilize the portion of the body to which the external support structure is separably attached using the apparatus 100 described herein. In some embodiments, the external support structure 150 may be passive, and in other embodiments, it may be active. In certain embodiments, the external support structure 150 may combine passive and active elements. In some embodiments, the external support structure 150 may comprise, for example, an articulated support structure, i.e., a support structure having one or more joints. In some embodiments, the articulated support structure may comprise one or more connected segments. In some embodiments, the segments may be pivotally connected or otherwise movably connected. In some embodiments, the external support structure 150 may be powered. A powered external support structure may comprise, for example, one or more motors. In some embodiments, the apparatus 100 described herein, when attached to the external support structure 150, permits the maximum range of motion in all planes and directions allowed by the external support structure itself.

### Support Member (Not Shown)

In some embodiments, the wearable component 110 may have a support member (not shown) affixed to its exterior surface. The support member may be affixed to the exterior surface using, for example, an adhesive, a glue, a combination of hook and loop surfaces, such as those sold under the trade name VELCRO®, or a combination of elastic straps. In some embodiments, the support member may be removably attached to the wearable component 110. In some embodiments, the support member may comprise a substantially rigid material, such as a metal or rigid polymer. In some embodiments, the support member may comprise a material selected from the group consisting of metal, ferrous metal, polyethylene terephthalate, polyethylene, polyvinyl chloride, polypropylene, polystyrene, fiberglass, carbon fiber, or any combination thereof. In some embodiments, the support member may further comprise a casing made from leather, synthetic leather, cloth, or any combination thereof. In some embodiments, the support member may be shaped to conform, or approximately conform, to a portion of a body.

### Engagement Element (Not Shown)

In some embodiments, one or more of the wearable component 110, the connecting portion 120, and the attachment element 130 may include one or more engagement elements (not shown). The engagement element (not shown) may be embedded into or affixed to the surface of one or more of the wearable component 110, the connecting portion 120, and the attachment element 130. In some embodiments, an engagement element (not shown) may include a clamp, a bar, a bracket, a clip, a quick coupling construct, a protrusion, a magnet, or any combination thereof. In some embodiments, the engagement element may include a magnet having an attractive force in the range from about 1 pound to about 40 pounds. In some embodiments, the attractive force of the magnet may be, for example, about 1 pound, about 2 pounds, about 3 pounds, about 4 pounds, about 5 pounds, about 6 pounds, about 7 pounds, about 8 pounds, about 9 pounds, about 10 pounds, about 11 pounds, about 12 pounds, about 13 pounds, about 14 pounds, about 15 pounds, about 16 pounds, about 17 pounds, about 18 pounds, about 19 pounds, about 20 pounds, about 21 pounds, about 22 pounds, about 23 pounds, about 24 pounds, about 25 pounds, about 26 pounds, about 27 pounds, about 28 pounds, about 29 pounds, about 30 pounds, about 31 pounds, about 32 pounds, about 33 pounds, about 34 pounds, about 35 pounds, about 36 pounds, about 37 pounds, about 38 pounds, about 39 pounds, about 40 pounds, or a range between any two of these values, including endpoints.

### Method of Stabilization

In some embodiments, a method of stabilization may include obtaining 200 an apparatus having a wearable component and a connecting portion, as described herein; applying 210 the wearable component to a portion of a body; attaching 220 an attachment element to an external support structure; and separably attaching 230 connecting portion of the wearable component to the attachment element of the external support structure.

In some embodiments, applying the wearable component to the portion of the body may include, for example, stretching or otherwise placing the wearable component, or a portion thereof, around the portion of the body, and adjusting it such that the wearable component is appropriately placed on and comfortable for the portion of the body. In some embodiments, applying the wearable component to the portion of the body may include applying a compressive force to the portion of the body. In some embodiments, the wearable component may itself apply a compressive force to the portion of the body when it is applied.

In some embodiments, the wearable component may include a strap having a hook-and-loop surface, and applying the wearable component to a portion of the body may include wrapping the strap around the portion of the body and separably attaching a first portion of the strap to a second portion of the strap using the hook-and-loop surface. In some embodiments, applying the wearable component to the portion of the body includes contacting the slip-resistant material of the wearable component with the portion of the body to create a coefficient of friction sufficient to resist substantial slipping between the wearable component and the portion of the body.

In some embodiments, applying the wearable component to the portion of the body may include placing the wearable component between the portion of the body and an article of clothing. In some embodiments, placing the wearable component between the portion of the body and an article of clothing may conceal, either partially or fully, the wearable component and the connecting portion affixed thereto.

What is claimed is:

1. An apparatus comprising:
a wearable component having an interior surface and an exterior surface;
a slip-resistant material adhered to the interior surface of the wearable component;
the wearable component separably attached to a connecting portion having a receiving element; and
the receiving element separably attached to an attachment element removably affixed to an external support structure.

2. The apparatus of claim 1, wherein the wearable component further comprises a strap having a hook-and-loop surface.

3. The apparatus of claim 1, wherein the wearable component and the connecting portion each independently comprise a polymer selected from the group consisting of an air-cured polymer, a temperature-cured polymer, polyethylene terephthalate, polyethylene, polyvinyl chloride, polypropylene, polystyrene, fiberglass, carbon fiber, and combinations thereof

4. The apparatus of claim 1 wherein the slip-resistant material comprises silicone.

5. The apparatus of claim 1, wherein the attachment element is selected from the group consisting of a clamp, a bar, a bracket, a clip, a protrusion, and combinations thereof.

6. The apparatus of claim 1, wherein the attachment element comprises a bar and a clamp.

7. The apparatus of claim 6, wherein the clamp is configured to slide along the length of the bar.

8. The apparatus of claim 1, wherein the external support structure comprises an articulated support structure comprising a plurality of pivotally connected segments.

9. The apparatus of claim 1, wherein one or more of the wearable component and the connecting portion are shaped to conform to a portion of a body.

10. The apparatus of claim 9, wherein the portion of the body is selected from the group consisting of a portion of an arm, a portion of a leg, a portion of a torso, a portion of a head, and a portion of a neck.

11. An apparatus comprising:
a wearable component having an interior surface and an exterior surface;
a slip-resistant material adhered to the interior surface of the wearable component;
the wearable component configured to separably attach to a connecting portion having a receiving element; and
the receiving element configured to receive an attachment element removably affixed to an external support structure.

12. A method of stabilization comprising:
obtaining an apparatus comprising a wearable component having an interior surface and an exterior surface, a slip-resistant material adhered to the interior surface of the wearable component, the wearable component separably attached to a connecting portion having a receiving element, and the receiving element configured to receive an attachment element removably affixed to an external support structure;
applying the wearable component to a portion of a body;
attaching the attachment element to the external support structure; and
separably attaching the connecting portion to the attachment element.

13. The method of stabilization of claim 12, wherein the wearable component further comprises a strap having a hook-and-loop surface, and wherein applying the wearable component to the portion of the body comprises wrapping the strap around the portion of the body and separably attaching a first portion of the strap to a second portion of the strap using the hook-and-loop surface.

14. The method of stabilization of claim 12, wherein applying the wearable component to the portion of the body comprises applying a compressive force to the portion of the body.

15. The method of stabilization of claim 12, wherein applying the wearable component to the portion of the body comprises placing the wearable component between the portion of the body and an article of clothing.

16. The method of stabilization of claim 15, wherein placing the wearable component between the portion of the body and the article of clothing at least partially conceals the wearable component.

17. The method of stabilization of claim 12, wherein applying the wearable component to the portion of the body comprises contacting the slip-resistant material with the portion of the body to create a coefficient of friction sufficient to resist substantial slipping between the wearable component and the portion of the body.
